(19) United States
(12) Patent Application Publication
(10) Pub. No.: US 2008/0058931 A1
(43) Pub. Date: Mar. 6, 2008

(54) Expandable vertebral implant and methods of use

(76) Inventors: John White, Bartlett, TN (US); Jason A. Edie, Memphis, TN (US)

Correspondence Address: COATS & BENNETT, PLLC, 1400 Crescent Green, Suite 300, Cary, NC, 27518, US

(21) Appl. No.: 11/491,466

(22) Filed: Jul. 21, 2006

(51) Int. Cl. A61F 2/44

(52) U.S. Cl. 623/17.11

(57) ABSTRACT

An implant for insertion between vertebral body endplates includes first and second end members, each with a respective endplate contact surface and an extension portion. An intermediate section may be contained between the first and second end members and includes a port into which a substance may be inserted. The intermediate section may be expandable upon the introduction of the substance between a first size to space the first and second end members a first distance apart and a second enlarged size to space the first and second end members a second greater distance apart. The intermediate section may be implemented as an expandable balloon-like member. The intermediate section may include a permeable portion to allow the substance, which may include bone growth materials, to pass from inside the implant to contact the vertebral body endplates.

### BACKGROUND

Spinal implants are often used in the surgical treatment of spinal disorders such as degenerative disc disease, disc herniations, scoliosis or other curvature abnormalities, and fractures. Many different types of treatments are used, including the removal of one or more vertebral bodies and/or intervertebral disc tissue. In some cases, spinal fusion is indicated to inhibit relative motion between vertebral bodies. In other cases, dynamic implants are used to preserve motion between vertebral bodies. In yet other cases, relatively static implants that exhibit some degree of flexibility may be inserted between vertebral bodies.

Regardless of the type of treatment and the type of implant used, surgical implantation tends to be a difficult for several reasons. For instance, access to the affected area may be limited by other anatomy. Further, a surgeon must be mindful of the spinal cord and neighboring nerve system. The size of the implant may present an additional obstacle. In some cases, a surgeon may discover that an implanted device has an inappropriate size for a particular application, which may require removal of the implant and insertion of a different implant. This trial and error approach may increase the opportunity for injury and is certainly time-consuming. Expandable implants are becoming more prevalent as a response to some of these concerns. However, the expansion mechanism in these devices tends to be complex and large. Consequently, existing devices do not appear to address each of these issues in a manner that improves the ease with which the device may be surgically implanted.

### SUMMARY

Illustrative embodiments disclosed herein are directed to an implant for insertion between vertebral body endplates. The implant may include first and second end members. Each endplate may include a respective bone-contact surface and an extension portion. The extension portions may engage each other in a sliding manner. An intermediate section may be contained between the first and second end members and includes a port into which a substance may be inserted. The intermediate section may be expandable upon the introduction of the substance. For instance, the intermediate section may expand between a first size to space the first and second end members a first distance apart and a second enlarged size to space the first and second end members a second greater distance apart. The intermediate section may be implemented as an expandable balloon-like member. The balloon-like member may be contained within a contiguous volume formed between the first and second end members. The balloon-like member may be a separate member or may be secured to the first and second end members. The intermediate section may include a permeable portion to allow the substance, which may include bone growth materials, to pass from inside the implant to contact the vertebral body endplates. For instance, the permeable portion, may be placed adjacent apertures in the bone-contact surfaces of the first and second end members. Accordingly, as the substance passes through the permeable portion, the substance further passes through the apertures and into contact with the vertebral bodies.

### BRIEF DESCRIPTION OF THE DRAWINGS

FIG. 1 is a side elevation view of a vertebral implant according to one embodiment positioned between vertebral bodies;

FIG. 2 is a section view of the vertebral implant according to the section lines in FIG. 1;

FIG. 3 is a perspective view of an exploded vertebral implant assembly according to one embodiment;

FIGS. 4-6 illustrate a sequence of implantation steps to obtain a desired vertebral body spacing, each Figure depicting a lateral view of a vertebral implant according to one or more embodiments shown relative to vertebral bodies;

FIG. 7 is a perspective view of an exploded vertebral implant assembly according to one embodiment;

FIG. 8 is a side section view of one embodiment of a vertebral implant in a collapsed state; and

FIG. 9 is a side view of a vertebral implant assembly according to one embodiment.

### DETAILED DESCRIPTION

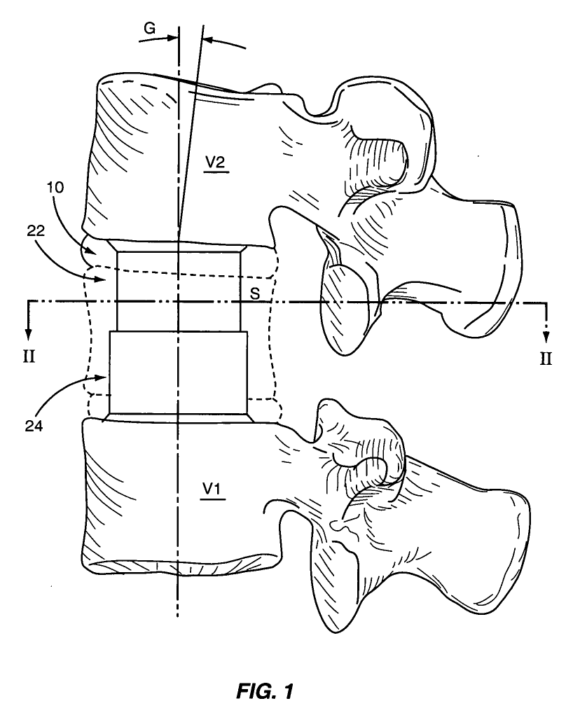

The various embodiments disclosed herein are directed to vertebral implants that are characterized by at least one expandable portion. The expandable portion may be compressed or left unfilled during installation of the implant and may be filled with an injectable substance once the implant is positioned within the body. An exemplary implant 10 for supporting vertebral bodies is illustrated in FIG. 1. In one embodiment, the implant 10 is a vertebrectomy or corpectomy cage assembly positionable within an intervertebral space to span one or more vertebral levels along the longitudinal axis of the spinal column. Although the illustrated embodiment of the implant 10 spans one vertebral level, it should be understood that the implant 10 may be configured to span multiple vertebral levels.

FIGS. 1-3 illustrate that the implant 10 generally includes a first end member 22, a second end member 24, and one or more expandable portions 26 between the first and second end members 22, 24. In one embodiment, the end members 22, 24 are formed of a biocompatible material, such as, for example, a carbon fiber material, or non-metallic substances, including polymers or copolymers made from materials such as PEEK and UHMWPE. In further embodiments, the end members 22, 24 may be formed of other suitable materials, such as, for example, stainless steel, titanium, cobalt-chrome, and shape memory alloys or other biocompatible metals.

The end members 22, 24 are adapted to engage the endplates of upper and lower vertebral bodies V1, V2. The expandable portion 26 is engaged between the end members 22, 24 to maintain an intervertebral axial space S between the upper and lower vertebral bodies V1, V2 following the removal of one or more vertebral levels (shown in phantom in FIG. 1). To facilitate insertion of the implant 10, the expandable portion 26 may be collapsed relative to the extended state shown in FIG. 1. Further details regarding process steps for insertion of the implant 10 are provided below.

The expandable portion 26 is expandable in a direction that is substantially transverse to the bone contact surfaces 32, 34 of the end members 22, 24. The bone contact surfaces 32, 34 of the end members 22, 24 may be planar or define surface features and/or a number of anchor elements 80 adapted for engagement with the vertebral endplates to inhibit movement of the end members 22, 24 relative to the vertebral bodies V1, V2. For example, in one embodiment, the bone contact surfaces 32, 34 may be roughened, such as, for example, by knurling and/or etching (e.g., photochemical etching). In other embodiments, various types of projections or protrusions may extend from the bone contact surfaces 32, 34, such as, for example, a number of spikes, ridges, teeth, axial grooves, checkerboard-type grooves, or any other type of anchoring element 80 that would occur to one of skill in the art. Although the bone contact surfaces 32, 34 of the end members 22, 24 are illustrated in FIG. 1 as being arranged substantially parallel to one another, it should be understood that the bone contact surfaces 32, 34 may be tapered relative to one another to more closely conform with the anatomical curvature of the spine at the surgical site (e.g., the angle of lordosis or kyphosis). It should also be understood that the end members 22, 24 may be positioned as desired with a predetermined curvature indicated by the angle G in FIG. 1 to more closely match the configuration of the implant 10 with the anatomical curvature of the spine at the surgical site.

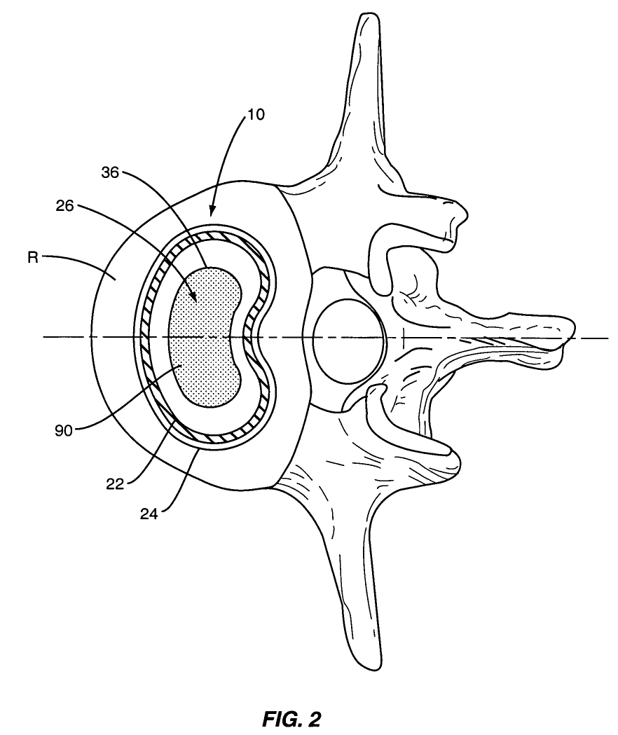

In one or more embodiments, the implant 10 may be expanded through the introduction of an injectable substance that fills an inflatable balloon-like member 36, thereby causing the end members 22, 24 to move opposite one another. The number 90 in FIG. 2 identifies the injectable substance, which fills the balloon-like member 36. In the embodiment shown, the expandable portion 26 includes a size and shape to fit within the end members 22, 24. The end members 22, 24 are illustrated as nested telescoping members in that end member 22 fits within and is slidably coupled to end member 24. When assembled as shown in FIG. 1, the end members 22, 24 form a cavity within which the expandable portion 26 is contained.

The end members 22, 24 include complementary shapes, which permits the end members 22, 24 to expand in a controlled manner. In the illustrated embodiment, the end members 22, 24 include a kidney shape, though other shapes may be used. In further embodiments, the end members 22, 24 may take on other types of configurations, such as, for example, a circular shape, semi-oval shape, bean-shape, D-shape, elliptical-shape, egg-shape, or any other shape that would occur to one of skill in the art. In other embodiments, the end members 22, 24 could also be described as being annular, U-shaped, C-shaped, V-shaped, horseshoe-shaped, semi-circular shaped, semi-oval shaped, or other similar terms defining an implant including at least a partially open or hollow construction. Thus, end members 22, 24 may be constructed for use in a variety of procedures, including but not limited to those requiring an anterior approach, a lateral approach, a posterior approach, or a trans-foraminal approach.

It should further be appreciated that the size and/or configuration of the end members 22, 24 may be specifically designed to accommodate any particular region of the spinal column and/or any particular vertebral level. For example, in embodiments associated with the upper thoracic or cervical region of the spine, the end members 22, 24 may be designed to have a D-shaped configuration, whereas embodiments associated with the lumbar region of the spine may be configured to have a horseshoe-shape, a U-shape, or other types of open-sided configurations.

In one embodiment, the end members 22, 24 have an outer profile that is substantially complementary to the size and shape of the peripheral portion or outlying region of the vertebral bodies V1, V2, such as the cortical rim or the apophyseal ring of the vertebral endplates. For example, as illustrated in FIG. 2, the outer perimeter of the end member 24 is preferably disposed generally above the inner edge of the cortical rim R of the vertebral body V1. In this manner, at least a portion of the end members 22, 24 is engaged against the cortical region of the vertebral endplates, thereby minimizing the likelihood of subsidence into the relatively softer cancellous region of the vertebral bodies V1, V2 following insertion of the implant 10 within the intervertebral space S. Additionally, each of the bone contact surfaces 32, 34 may include apertures or recesses 82 to enhance bony fusion between the end members 22, 24 and vertebral bodies V1, V2. The recesses 82 may be blind holes in that they do not extend through the end members 22, 24 and into the interior cavities 50A, 50B. The recesses 82 may be through-holes in that they do extend through the end members 22, 24 and into cavities 50A, 50B. In one or more implementations, the implant 10 may be inserted in conjunction with bone growth materials that may include, for example, bone graft, bone morphogenetic protein (BMP), allograft, autograft, and various types of cement, growth factors and mineralization proteins. In a further embodiment, the bone growth promoting materials may be provided in a carrier (not shown), such as, for example, a sponge, a block, a cage, folded sheets, or paste. The bone growth materials may be loaded into the apertures 82 or generally applied to the bone-contact surfaces 32, 34.

The bone-contact surfaces 32, 34 are disposed at a base portion 38, 40 of the end members 22, 24, respectively. An extension portion 42, 44 protrudes from the base portions 38, 40 in each end member 32, 34. The extension portions 42, 44 include a peripheral wall 46, 48 with complementary shapes. In the illustrated embodiment, the peripheral walls 46, 48 form a kidney shape similar to the base portion 38, 40, however this is not expressly required. The peripheral walls 46, 48 may be cylindrical, rectangular, triangular, or any other suitable shape that would occur to one skilled in the art. The peripheral walls 46, 48 form respective interior cavities 50A, 50B that form a contiguous cavity 50 when the end members 22, 24 are coupled to one another.

The balloon-like structure 36 may be constructed of a complaint biocompatible material, such as a resin or polymer that may include materials such as nylon, polyethylene, polyurethane, silicone, polyethylene, polypropylene, polyimide, polyamide, and polyehteretherketone (PEEK). The balloon-like structure 36 may be formed from materials that are used in other conventionally known biomedical applications, such as balloon angioplasty. Further, the balloon-like structure 36 may be reinforced with concentric layers of similar or dissimilar materials and/or fabrics (not specifically shown). For instance, a reinforcing structure may be constructed of a wide variety of woven or nonwoven fibers, fabrics, metal mesh such as woven or braided wires, polymeric fibers, ceramic fibers, and carbon fibers. Biocompatible fabrics or sheet material such as ePTFE and Dacron®, Spectra®, and Kevlar® may also be used. Furthermore, the balloon-like structure 36 may be a separate member or may be secured to one or both of the end members 22, 24.

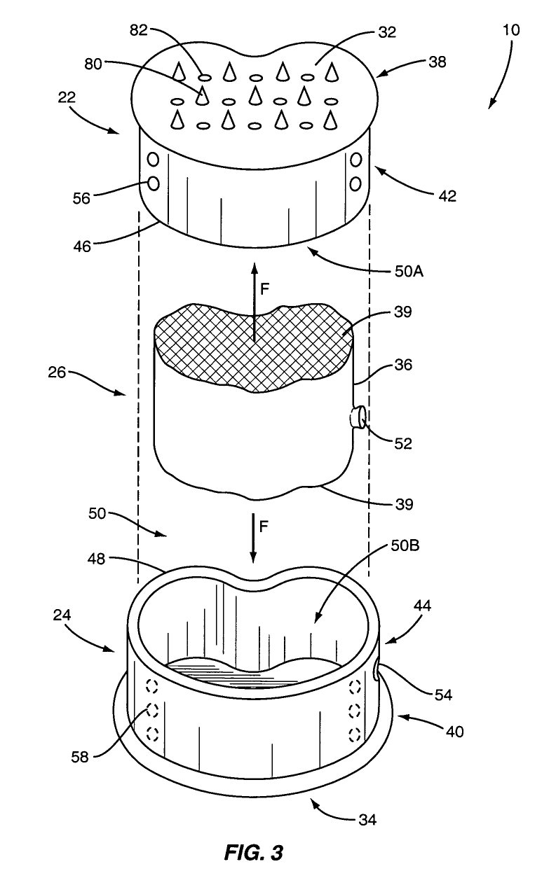

Various techniques may be used to introduce an injectable substance into the balloon-like structure 36. In the embodiment shown, a fill port 52 extends from the balloon-like structure 36. FIG. 3 most clearly shows that the fill port 52 provides a duct that is in fluid communication the interior of the balloon-like structure 36. Notably, while only one fill port 52 is depicted, additional ports 52 may be used. Further, the port 52 may be located in different locations depending on a particular implementation and angle of approach. In the embodiment shown, end member 24 includes a fill aperture 54 that includes a corresponding size and location to provide access to the fill port 52 when the balloon-like structure 36 is positioned within the end member 24. The fill port 52 may be attached to a syringe or other pumping mechanism (not shown) to fill the balloon-like structure 36. An injectable substance may flow through the fill port 52 into the interior volume of the balloon-like structure 36. As the injectable substance fills the balloon-like structure 36, the ends 39 of the balloon-like structure 36 extend through cavities 50A, 50B and expand to fill the overall cavity 50 formed within the end members 22, 24. As the ends 39 of the balloon-like structure 36 expand, they exert a displacement force F that causes the end members 22, 24 to separate from one another. Furthermore, the fill tube 42 or the coupler 44 may include a self-sealing valve (not specifically shown) that prevents the injectable substance from flowing in one direction or another once the balloon-like structure 36 is filled.

A variety of injectable substances may be inserted into the balloon-like structure 36 to cause the end members 22, 24 to separate. In one embodiment, the injectable substance is a fluid, such as a gas or a liquid. In one embodiment, the injectable substance is a solid, such as a powder. In one embodiment, the injectable substance is a curable liquid that solidifies after a predetermined amount of time or under the influence of an external catalyst. For instance, an injectable liquid may cure under the influence of heat or light, including ultraviolet light. Some examples of in situ curable liquids include epoxy, PMMA, polyurethane, and silicone. A curable substance may cure to a substantially rigid state or to a flexible, but relatively incompressible state.

In certain implementations, where the injectable substance remains fluid or takes an extended period of time to cure, the end members 22, 24 are provided with position locks 56, 58. In one embodiment, end member 22 includes protruding features 56 disposed at various heights about the exterior of the peripheral wall 46. Correspondingly, end member 24 includes recessed features 58 disposed at various heights about the interior of the peripheral wall 48. Thus, when the end members 22, 24 are joined to one another, the protruding features 56 engage the recessed features 58 to provide a locked height that prevents compression of the implant 10. That is, as the balloon-like structure 36 is filled with an injectable substance, the end members 22, 24 will separate and expand to a position where a protrusion 56 engages a recess 58. At this point, introducing additional injectable substance will force the protrusion 56 to disengage from the recess 58 and ultimately engage a next higher recess 58. The protrusions 56 and/or the recesses 58 may be angled, tapered, or oriented to permit expansion of the implant 10 but not compression in the reverse direction. Those skilled in the art will comprehend a variety of ways to implement this type of unidirectional locking.

The protruding features 56 may be implemented using a variety of features, including but not limited to ball plungers, expanding pegs, protruding stops, and shape-memory alloys. In the latter case, the protruding features 56 may be positioned in a first retracted position and then, upon the application of elevated temperatures (which may be provided by body temperatures), the protruding feature 56 will expand to engage a recess 58 corresponding to a desired implant height.

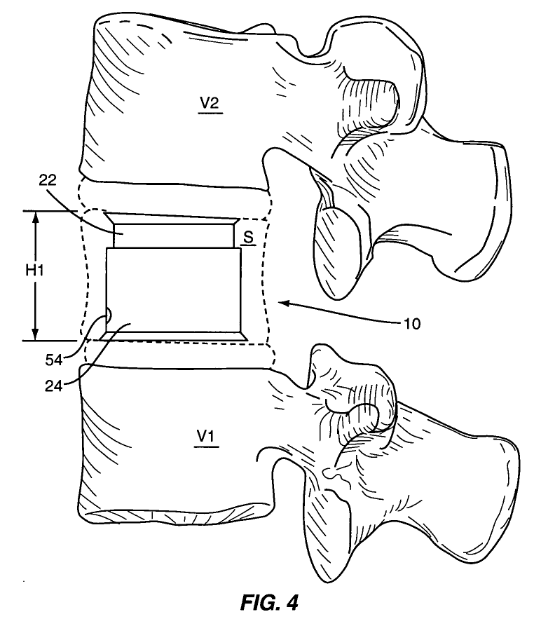

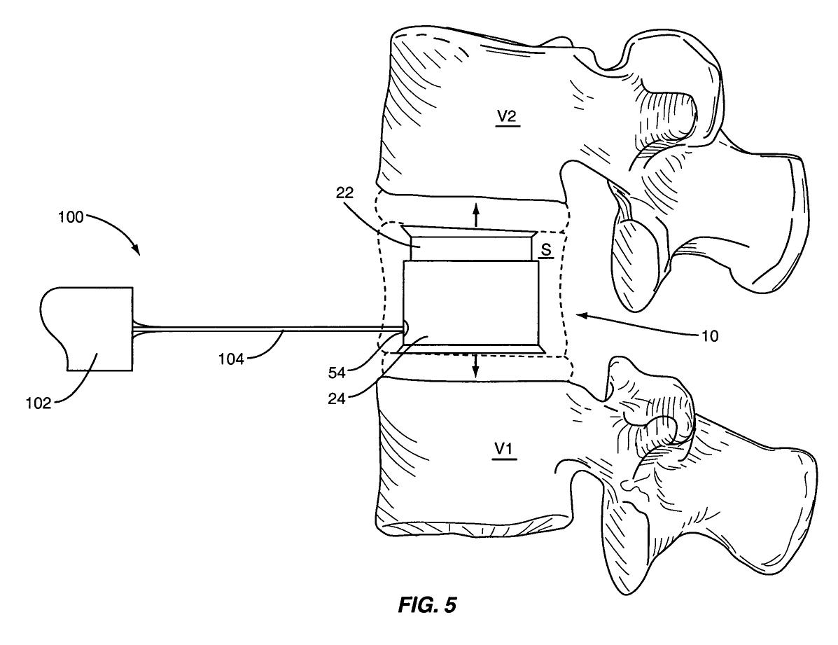

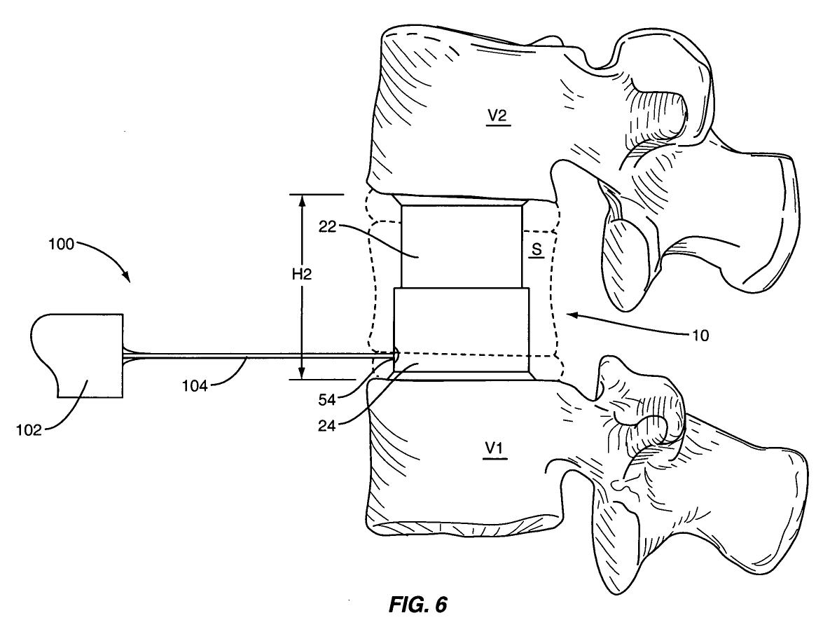

The implant 10 may be inserted into a patient according to the process steps illustrated in FIGS. 4-6. In FIG. 4, the implant 10 is inserted in a compressed first state including a first height H1 and positioned within an intervertebral space formed after the removal of one or more vertebrae or discs. Once the implant 10 is positioned as shown in FIG. 5, the inflation tool 100 or other injection instrument is used to inject the injectable substance into the fill port 52 on the balloon-like structure 36. As described above, access to the fill port 52 is provided through a fill aperture 54 in the end member 24. The inflation tool 100 may be implemented as a syringe-like structure including a reservoir portion 102 and a delivery portion 104. The delivery portion 104 is configured to engage the fill port 52 to transfer the injectable substance from the reservoir portion 102 into the balloon-like structure 36. Other delivery mechanisms are certainly appropriate. For instance, pneumatic or hydraulic fittings may be appropriate. The delivery portion 104 may be implemented as a needle, as tubing, or other cannulated devices. In any event, as the injectable substance is introduced into the implant 10, the end members 22, 24 are forced apart due to the expansion of the contained balloon-like structure 36. Ultimately, the implant is expanded to an expanded second state including a second height H2 as shown in FIG. 6.

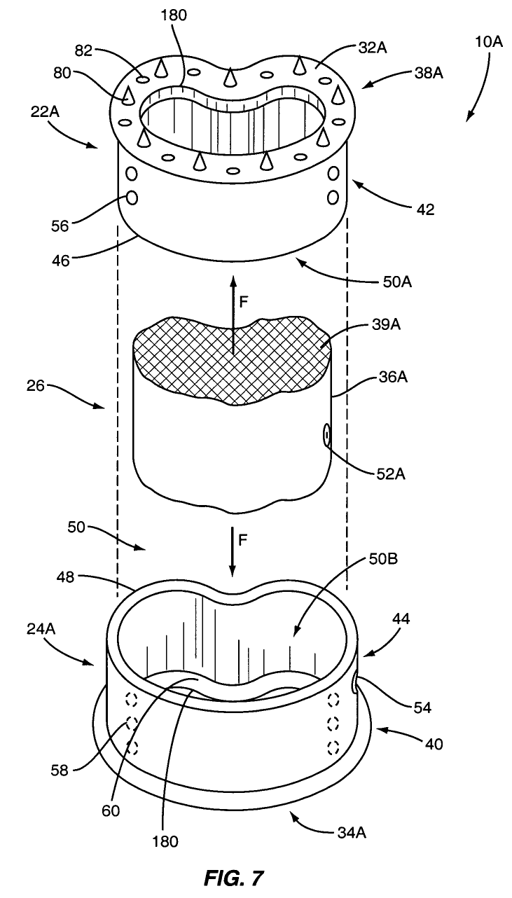

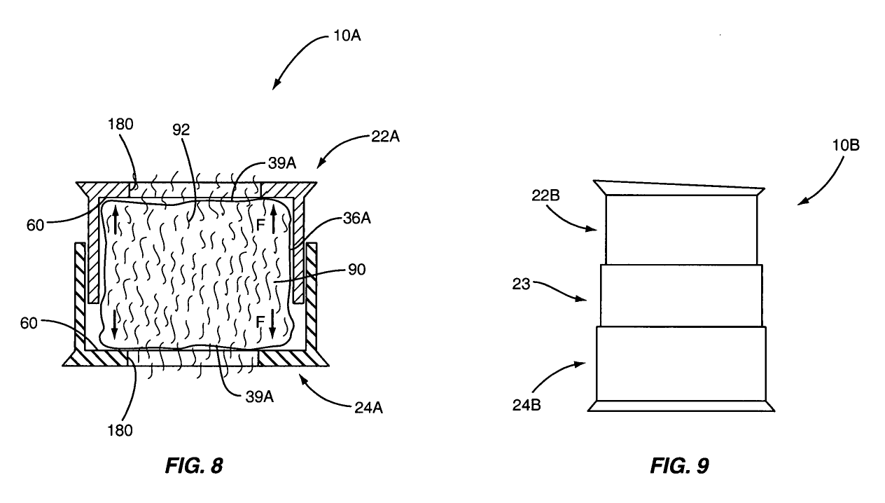

FIGS. 7 and 8 depict an embodiment of an implant 10A in which the end members 22A, 24A include one or more enlarged apertures 180 that extend from the bone contact surfaces 32A, 34A to the interior cavities 50A, 50B. FIG. 7 shows an exploded assembly view of the implant 10A while FIG. 8 shows a lateral cross section of the assembled implant 10A. The apertures 180 may extend over some or most of the bone contact surfaces 32A, 34A. However, as FIG. 8 illustrates, the apertures 180 are sized and positioned to provide a sufficient contact surface 60 within the cavities 50A, 50B against which the balloon-like structure 36A exerts an expansion force indicated by the arrows F. Notably, the implant 10A further includes a fill port 52A that is substantially flush with the outer surface of the balloon-like structure 36A.

Furthermore, the present embodiment includes a balloon-like structure 36A that includes permeable end surfaces 39A. That is, the end surfaces 39A include a perforated, grated, or mesh-like structure that allows the injectable substance 90 to pass from within the balloon-like structure 36A and through the apertures 180 to contact the corresponding vertebral bodies V1, V2 (see e.g., FIG. 1). In one embodiment, most or all of the balloon-like structure 36A is permeable in a similar manner. The permeable nature of at least the end surfaces 39A makes it advantageous to include bone growth promoting materials within the injectable substance. The number 92 in FIG. 8 generally represents the growth promoting materials that are suspended in the injectable substance 90. Accordingly, as the injectable substance 90 is inserted into the balloon-like structure 36A, the end members 22A, 24A will expand under the influence of the expansion force F provided by the balloon-like structure 36. Additionally, some of the injectable substance 90 will exit the permeable end surfaced 39A and fill the apertures 180. Consequently, the growth promoting materials 92 are positioned to enhance bone growth from adjacent vertebral bodies V1, V2 into the implant 10A. In one embodiment, the permeable end surfaces 39A may be configured to contain the injectable substance 90 until a certain internal pressure is obtained. Beyond that pressure, obtained through introducing additional injectable substance 90, the injectable substance 90 will exit the end surfaces 39A and enter the apertures 180.

Embodiments described above have generally included a first end member 22, 22A slidingly coupled to a second end member 24, 24A. It should be clarified, however, that the implant 10 may include additional intermediate members 23 as shown in the implant 10B depicted in FIG. 9. The intermediate member 23 may include a size and shape that permits the implant 10 to telescope when expanding under the influence of an expanding balloon-like member 36 contained therein. The addition of one or more intermediate members 23 may allow the implant to assume a shorter compressed height and/or a taller expanded height. Two or more intermediate members 23 may be used as appropriate.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. For instance, the embodiments disclosed herein have contemplated a single implant positioned between vertebral bodies V1, V2. In other embodiments, two or more smaller implants may be inserted between the vertebral bodies V1, V2. The present embodiments are, therefore, to be considered in all respects as illustrative and not restrictive, and all changes coming within the meaning and equivalency range of the appended claims are intended to be embraced therein.

1. An implant for insertion between vertebral body endplates in a patient, the implant comprising:
a first end member including a first endplate contact surface and a first extension portion;
a second end member including a second endplate contact surface and a second extension portion, the second extension portion slidingly engaged to the first extension portion;
an intermediate section contained between the first and second end members; and
a port operatively connected to the intermediate section;
the intermediate section being axially expandable upon the introduction of a substance into the port, the intermediate section being expandable between a first size to space the first and second end members a first distance apart and a second enlarged size to space the first and second end members a second greater distance apart.

2. The implant of claim 1 wherein the first and second extension portions remain slidingly engaged while the first and second end members expand between the first and second distances apart.

3. The implant of claim 1 wherein the first and second extension portions form a contiguous volume in which the intermediate section is contained.

4. The implant of claim 1 wherein the first and second endplate contact surfaces include an aperture that is open to the intermediate section.

5. The implant of claim 1 wherein the intermediate section is an inflatable balloon.

6. The implant of claim 1 wherein the intermediate member is connected to the first and second end members.

7. The implant of claim 1 wherein the first and second end members include position locks disposed at predetermined heights to prevent compression of the implant.

8. An implant for insertion between vertebral body endplates in a patient, the implant comprising:
a first end member including a first endplate contact surface a first interior volume;
a second end member including a second endplate contact surface and a second interior volume;
an inflatable intermediate member connected to the first and second end members and sized to fit within the first and second volumes, the intermediate member including a permeable portion; and
a port operatively connected to the intermediate member;
the intermediate member being axially expandable upon the introduction of a substance into the port, the intermediate section being expandable between a first size to space the first and second end members a first distance apart and a second enlarged size to space the first and second end members a second greater distance apart, the permeable portion allowing the substance to pass from inside the intermediate member to outside the intermediate member.

9. The implant of claim 8 wherein the first and second endplate contact surfaces include an aperture that is open to the first and second interior volumes.

10. The implant of claim 9 wherein the permeable portion is disposed in proximity to the apertures so the substance that passes from inside the intermediate member to outside the intermediate member passes into the apertures.

11. The implant of claim 8 wherein substantially all of the intermediate member is permeable.

12. The implant of claim 8 wherein the first and second end members form a contiguous volume in which the intermediate member is contained.

13. The implant of claim 8 wherein the first and second end members and the intermediate member are separate members.

14. The implant of claim 8 wherein the first and second end members include position locks disposed at predetermined heights to prevent compression of the implant.

15-25. (canceled)

26. An implant for insertion between vertebral body endplates in a patient, the implant comprising:
an exterior section comprising first and second members in a telescoping arrangement that each include a contact surface positioned at an axial end of the exterior section;
a port extending through the exterior section;
an inflatable intermediate section positioned within an interior of the exterior section, the intermediate section including an inlet aligned with the port to receive a fill material;
the intermediate section being axially expandable from a first size prior to insertion of the fill material that spaces the contact surfaces of the exterior section a first distance apart and a second size after insertion of the fill material that spaces the contact surfaces a second, greater distance apart.

27. The implant of claim 26, wherein each of the first and second members further comprises extensions that extend outward in one direction from the contact surfaces and are positioned in an overlapping arrangement when the intermediate section is in the first size.

28. The implant of claim 26, wherein each of the contact surfaces includes apertures such that the intermediate section is exposed to the vertebral bodies.

29. The implant of claim 28, wherein the intermediate section is constructed of a permeable material.

30. An implant for insertion between vertebral body endplates in a patient, the implant comprising:
an exterior section comprising a first end member including a first endplate contact surface and a second end member including a second end plate contact surface, the first and second end members being operatively connected to form
an interior section;
a port within the exterior section;
an inflatable member positioned within the interior section and including an inlet that aligns with the port;
the inflatable member being axially expandable upon introduction of a fill substance into the port and the inlet, the inflatable member being expandable between a first size to space apart the first and second contact surfaces a first distance and a second enlarged size to space apart the first and second contact surfaces a second greater distance.

31. The implant of claim 30, wherein the exterior section includes apertures to allow the inflatable member to contact the vertebral body endplates.

32. The implant of claim 30, wherein the first and second end members each include an extension, the first and second end members being in a telescoping arrangement with the extensions overlapping a first amount when the inflatable member is the first size and overlapping a second lesser amount when the inflatable member is the second size.
